(19) United States
(12) Patent Application Publication
(10) Pub. No.: US 2017/0135644 A1
(43) Pub. Date: May 18, 2017

(54) PHYSIOLOGICAL SIGNAL MEASURING SYSTEM AND METHOD THEREOF

(71) Applicant: NATIONAL CHIAO TUNG UNIVERSITY, Hsinchu City (TW)

(72) Inventors: Tzu-Chien HSIAO, Hsinchu City (TW); Sheng-Chi KAO, Taichung City (TW)

(21) Appl. No.: 15/052,896

(22) Filed: Feb. 25, 2016

(30) Foreign Application Priority Data
Nov. 18, 2015 (TW) 104138064

(51) Int. Cl. A61B 5/00; A61B 5/021; A61B 5/08

(52) U.S. Cl. CPC A61B 5/7246; A61B 5/0816; A61B 5/02108; A61B 5/6823; A61B 5/6824

(57) ABSTRACT

The physiological signal measuring method includes the following steps: obtaining a user physiological signal; separating the user physiological signal into multiple first packets according to a first box number; performing a sifting process respectively on the first packets by Empirical Mode Decomposition (EMD) to obtain multiple temporal intrinsic mode functions (temporal IMFs) respectively corresponding to the first packets; calculating multiple average envelope curves according to multiple upper envelope curves and multiple lower envelope curves respectively corresponding to the temporal IMFs; averaging the average envelope curves to generate a semi-IMF; calculating at least one correlation coefficient according to the semi-IMF and at least another semi-IMF; when the at least one correlation coefficient is larger than a correlation coefficient threshold, determining at least one signal section corresponding to the at least one correlation coefficient as at least one main component section of the user physiological signal.

### RELATED APPLICATIONS

This application claims priority to Taiwanese Application Serial Number 104138064, filed Nov. 18, 2015, the entirety of which is herein incorporated by reference.

### BACKGROUND

Field of Invention

The present invention relates to a physiological signal measuring system, and particularly to a physiological signal measuring system capable of processing signals in parallel and a method thereof.

Description of Related Art

In recent years, the measurement of physiological signals has a certain necessity in medical research. Generally, many physiological signals are not stable in waveform, such as physiological signals obtained by measuring a cardiovascular system and a respiratory system of the human body. It is difficult to analyze these physiological signals through a conventional method, and it is also difficult to present the information that varies over time. Therefore, in the conventional technology, a physiological signal is separated into multiple intrinsic mode functions (IMFs) by utilizing empirical mode decomposition (EMD), so as to solve the problem that the conventional Fourier spectrum may lose information under time variation.

However, in respect of real-time monitoring of physiological signals of a patient and emergency treatment or far-end micro nursing hardware, the treating process of the EMD may inhibit the coping and treatment speed and the possibility of portability. Even if an EMD calculating method in the conventional technology has a high decomposing capability, it is difficult to carry out parallel processing, so that the calculated amount and the calculation time cannot be reduced, and the application value is reduced. Besides, the EMD itself has the problems of failure in parallel calculation or inaccuracy in processing.

Therefore, it has become one of important issues in the field at present how to effectively and accurately analyze main components in physiological signals.

### SUMMARY

To solve the above-mentioned problem, an aspect of the disclosure provides a physiological signal measuring system. The physiological signal measuring system includes a processor. The processor includes a packeting module, an empirical mode decomposition module, an intrinsic mode function module and a main component module. The packeting module is used for obtaining a user physiological signal and separating the user physiological signal into multiple first packets according to a first box number. The empirical mode decomposition module is used for performing a sifting process respectively on the first packets by utilizing empirical mode decomposition (EMD), so as to obtain multiple temporal intrinsic mode functions (temporal IMFs) respectively corresponding to the first packets. The intrinsic mode function module is used for calculating multiple average envelope curves according to multiple upper envelope curves and multiple lower envelope curves respectively corresponding to the temporal intrinsic mode functions, and averaging the average envelope curves to generate a semi-intrinsic mode function (semi-IMF). The main component module is used for calculating at least one correlation coefficient according to the semi-intrinsic mode function and at least another semi-intrinsic mode function, and when the at least one correlation coefficient is larger than a correlation coefficient threshold, the main component module determines at least one signal section corresponding to the at least one correlation coefficient as at least one main component section of the user physiological signal.

Another aspect of the present invention provides a physiological signal measuring method. The physiological signal measuring method physiological signal measuring method includes the following steps: obtaining a user physiological signal and separating the user physiological signal into multiple first packets according to a first box number; performing a sifting process respectively on the first packets by utilizing empirical mode decomposition, so as to obtain multiple temporal intrinsic mode functions respectively corresponding to the first packets; calculating multiple average envelope curves according to multiple upper envelope curves and multiple lower envelope curves respectively corresponding to the temporal intrinsic mode functions; averaging the average envelope curves to generate a semi-intrinsic mode function; calculating at least one correlation coefficient according to the semi-intrinsic mode function and at least another semi-intrinsic mode function; and when the at least one correlation coefficient is larger than a correlation coefficient threshold, determining at least one signal section corresponding to the at least one correlation coefficient as at least one main component section of the user physiological signal.

In view of the above, compared with the prior art, the technical solution of the present invention has obvious advantages and beneficial effects. With the aforementioned technical solution, a considerable technical progress can be achieved with the value of being widely applied in the industry. By means of the physiological signal measuring system capable of processing signals in parallel and the method thereof according to the disclosure, the signal is decomposed into the multiple packets after a packeting process, and thus the multi-core processor can be utilized to process the packets in parallel, so as to improve the processing speed. In addition, after the positions of main components are found for the first time, the decomposition can be performed directly according to the packet number of the main components in the follow-up process, so as to greatly reduce the calculated amount in the decomposition process in a conventional method.

### BRIEF DESCRIPTION OF THE DRAWINGS

FIG. 1 illustrates a block diagram of a physiological signal measuring system according to an embodiment of the present invention;

FIG. 2 illustrates a schematic view of a mode of application of the physiological signal measuring system according to an embodiment of the present invention;

FIG. 3 illustrates a flow diagram of a physiological signal measuring method according to an embodiment of the present invention;

FIGS. 4A-4C illustrate schematic views of performing a packeting process on a user physiological signal according to an embodiment of the present invention;

FIG. 5 illustrates a schematic view of a correlation coefficient according to an embodiment of the present invention;

FIGS. 6A-6C illustrate schematic views of a mode of application of the physiological signal measuring system according to an embodiment of the present invention; and

FIG. 7 illustrates a flow diagram of a physiological signal measuring method according to an embodiment of the present invention.

### DETAILED DESCRIPTION

The present invention is described in detail in the following embodiments with reference to the accompanying drawings. However, the embodiments provided are not intended to limit the scope of the present invention, and the description of the structural operation is not intended to limit the order of implementation of the operation. Any device with equivalent functions that is produced from a structure formed by a recombination of elements shall fall within the scope of the present invention. Furthermore, the drawings are illustrated only for purpose of illustration and are not drawn to scale. For convenience in understanding, the same elements are represented by the same reference numbers in the following description.

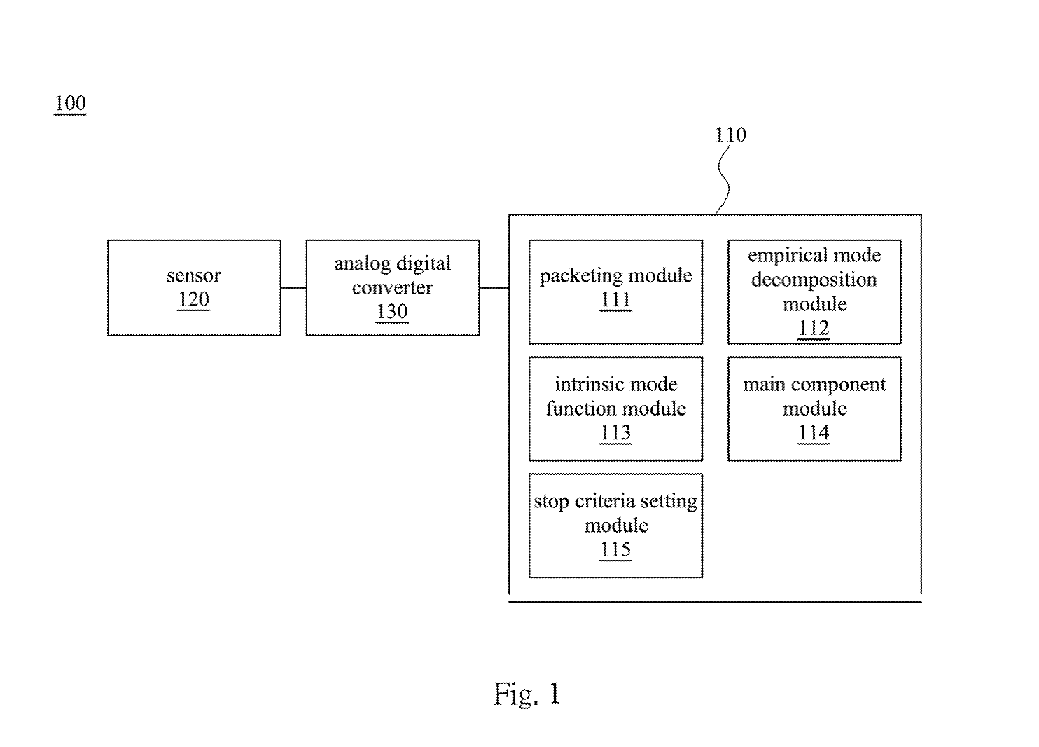

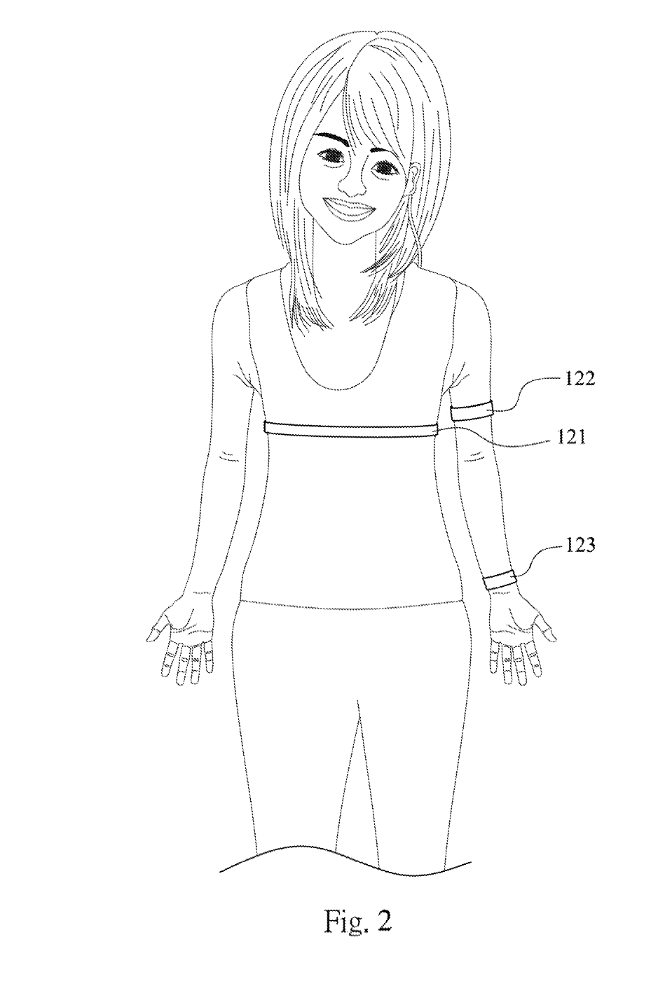

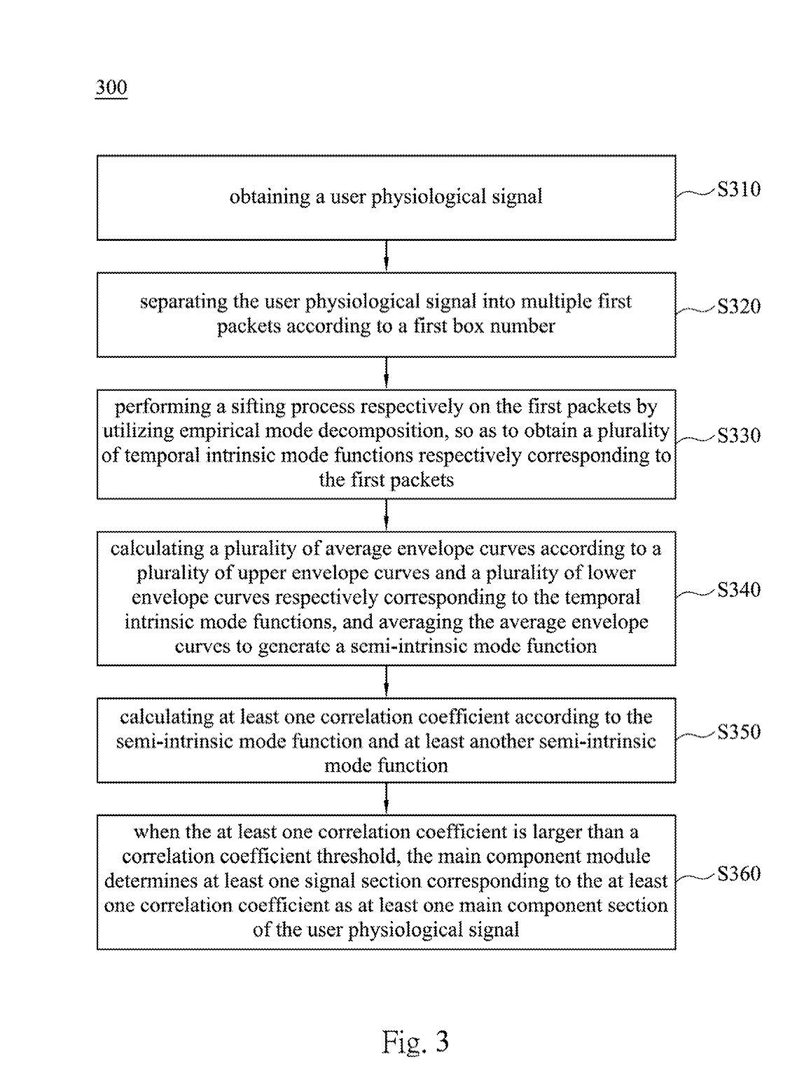

Referring to FIGS. 1-3, FIG. 1 illustrates a block diagram of a physiological signal measuring system 100 according to an embodiment of the present invention. FIG. 2 illustrates a schematic view of a mode of application of the physiological signal measuring system 100 according to an embodiment of the present invention. FIG. 3 illustrates a flow diagram of a physiological signal measuring method 300 according to an embodiment of the present invention.

As shown in FIG. 1, the physiological signal measuring system 100 includes a processor 110. The processor 110 includes a packeting module 111, an empirical mode decomposition module 112, an intrinsic mode function module 113 and a main component module 114. In one embodiment, the physiological signal measuring system 100 further includes a sensor 120 and an analog digital converter 130. Herein, the processor 110 further includes a stop criteria setting module 115. In one embodiment, the processor 110 can be a multi-core processor.

In the processor 110, the packeting module 111, the empirical mode decomposition module 112, the intrinsic mode function module 113, the main component module 114 and the stop criteria setting module 115 can be embodied independently or in combination through a volume circuit, such as a micro controller, a microprocessor, a digital signal processor, an application specific integrated circuit (ASIC) or a logic circuit.

In one embodiment, a physiological signal can be measured through the sensor 120 worn on the human body, such as a respiration sensor 121 worn on the abdomen, a blood pressure pulse sensor 122 worn on the arm or/and a blood pressure pulse sensor 123 worn on the wrist as shown in FIG. 2. However, those of ordinary skills in the art may understand that the present invention is not limited to these sensors.

Next, referring to FIG. 3, the present invention provides a physiological signal measuring method 300 for extracting important physiological information by means of parallelizable fractal empirical mode decomposition (FEMD). The physiological signal measuring method 300 is described below in detail.

In step S310, the packeting module 111 is used for obtaining a user physiological signal. In one embodiment, the user physiological signal can be measured through the sensor 120 worn on the human body, and the measured user physiological signal is transmitted to the packeting module 111, so that the packeting module 111 obtains the user physiological signal.

In step S320, the packeting module 111 separates the user physiological signal into multiple first packets according to a first box number.

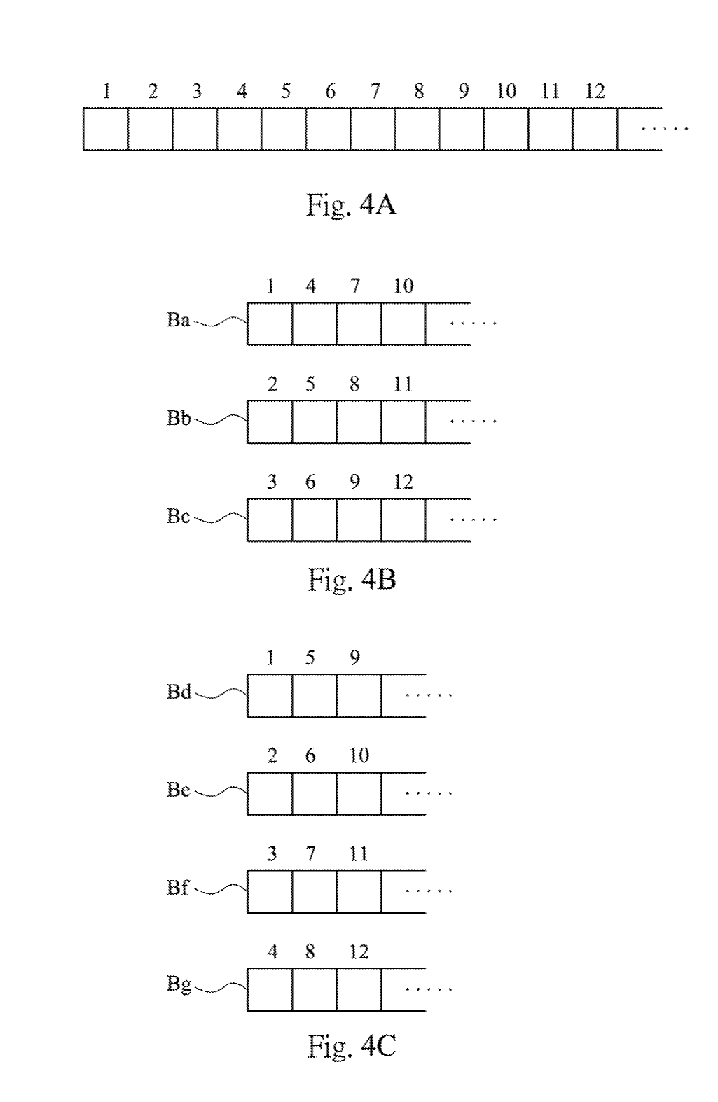

In one embodiment, after the packeting module 111 obtains the user physiological signal, on the conditions that the first box number is preset to be 3, the packeting module 111 separates the user physiological signal into three packets according to the first box number, such as packets Ba, Bb and Bc in FIG. 4B. In this embodiment, the packets Ba, Bb and Bc can be referred to as first packets. The method of performing a packeting process on the user physiological signal is further described below.

Referring to FIGS. 4A-4C, FIGS. 4A-4C illustrate schematic views of performing a packeting process on a user physiological signal according to an embodiment of the present invention. In one embodiment, the sensor 120 is used for measuring an initial physiological signal, such as a respiration signal, a pulse signal or a heartbeat signal, and the initial physiological signal is an analog signal. The analog digital converter 130 is used for converting the initial physiological signal into a user physiological signal, and the user physiological signal is a digital signal. Therefore, the packeting module 111 can cut the user physiological signal into multiple section signals 1-12 as shown in FIG. 4A, and each of the section signals 1-12 is a small section of digital signal simulated into an analog signal. For example, the section signal 1 is provided with a sub-signal of the user physiological signal from 0 second to 0.5 second, and the section signal 2 is provided with a sub-signal of the user physiological signal from 0.5 second to 1 second.

In one embodiment, after the packeting module 111 sequentially divides the user physiological signal into multiple section signals 1-12, the first packets Ba, Bb and Bc are formed by performing sequential decimation according to the section signals 1-12.

In one embodiment, as shown in FIG. 4B, on the conditions that the first box number is preset to be 3, the user physiological signal is divided into three first packets Ba, Bb and Bc. Herein, the first packet Ba is formed by section signals 1, 4, 7 and 10 with a section signal interval of 3; the first packet Bb is formed by section signals 2, 5, 8 and 11 with a section signal interval of 3; and the first packet Bc is formed by section signals 3, 6, 9 and 12 with a section signal interval of 3. It should be noted that the first packets Ba, Bb and Bc can have different lengths respectively according to the number of the section signals.

In one embodiment, as shown in FIG. 4C, on the conditions that the first box number is preset to be 4, the user physiological signal is divided into four packets Bd, Be, Bf and Bg. In this embodiment, the packets Bd, Be, Bf and Bg can be referred to as first packets. Herein, the first packet Bd is formed by section signals 1, 5 and 9 with a section signal interval of 4; the first packet Be is formed by section signals 2, 6 and 10 with a section signal interval of 4; the first packet Bf is formed by section signals 3, 7 and 11 with a section signal interval of 4; and the first packet Bg is formed by section signals 4, 8 and 12 with a section signal interval of 4. It should be noted that the physiological signal measuring system 100 can automatically or manually adjust the first box number according to the practical situation, so as to adopt the first box number most suitable for analyzing the user physiological signal.

By means of the above-mentioned step related with the packeting process, the user physiological signal can be decomposed into multiple first packets. Besides, by adopting a sequential decimation method, on one hand, the problem that the discontinuity surface is generated during the decomposition of the user physiological signal can be solved, and on the other hand, the calculated result has better local tendency.

In step 330, the empirical mode decomposition module 112 respectively performs a sifting process on the first packets by utilizing empirical mode decomposition (EMD), so as to obtain multiple temporal intrinsic mode functions (temporal IMFs) respectively corresponding to the first packets.

For example, in FIG. 4B, each of the first packets Ba, Bb and Bc performs signal decomposition respectively by utilizing the empirical mode decomposition, so as to obtain multiple temporal intrinsic mode functions respectively corresponding to the first packets.

It should be noted that the empirical mode decomposition was put forward by Norden E. Huang et al. in 1998. Through the empirical mode decomposition, a to-be-analyzed signal can be decomposed into intrinsic mode functions, and then the intrinsic mode functions undergo Hilbert transform, so as to correctly obtain instantaneous frequency of data. The method is used to process unsteady-state and nonlinear signals. The technical content of the sifting process is one link of the empirical mode decomposition, and thus it is not repeated herein. By applying the empirical mode decomposition to step S330 in the present invention, the sifting process is respectively performed on the multiple first packets, and multiple intrinsic mode functions obtained during the stage are defined as temporal intrinsic mode functions. However, those of ordinary skills in the art may understand that the application of the empirical mode decomposition in the step S330 is only part of the present invention and should not be regarded as the whole of the present invention.

In one embodiment, each time the empirical mode decomposition module 112 obtains a temporal intrinsic mode function by decomposition, the stop criteria setting module 115 judges whether a sifting result of the sifting process meets one stop criteria. Herein, the sifting result corresponds to one of the first packets (for example, one sifting result corresponds to the first packet Ba among the multiple first packets Ba, Bb and Bc). If the stop criteria setting module 115 judges that the sifting result of the sifting process meets the stop criteria, one of the temporal intrinsic mode functions is generated. In contrast, if the stop criteria setting module 115 judges that the sifting result of the sifting process does not meet the stop criteria, the sifting result is substituted into the empirical mode decomposition to perform the sifting process again.

For example, in FIG. 4B, a sifting result is generated after the sifting process is performed on the first packet Ba, and the stop criteria setting module 115 judges whether the sifting result of the sifting process meets one stop criteria. If the stop criteria setting module 115 judges that the sifting result of the sifting process meets the stop criteria, a temporal intrinsic mode functions is generated, and the sifting process on the next first packet Bb is performed. If the stop criteria setting module 115 judges that the sifting result of the sifting process does not meet the stop criteria, the current sifting result (namely the current sifting result of the first packet Ba) is substituted into the empirical mode decomposition again so as to continue performing the sifting process.

In one embodiment, the stop criteria is that the sum of a local maxima and a local minima must be equal to the number of zero crossings or can only differ by 1 at most. That is, one extremum must be followed by a zero crossing at once, and at any time point, the average of an upper envelope curve defined by the local maxima and a lower envelope curve defined by the local minima should approach 0. The technical content of the stop criteria is one link of the empirical mode decomposition, and thus it is not repeated herein.

Therefore, in the example shown in FIG. 4B, the empirical mode decomposition module 112 can respectively perform the sifting process on the multiple first packets Ba, Bb and Bc by utilizing empirical mode decomposition, so as to obtain multiple temporal intrinsic mode functions respectively corresponding to the first packets Ba, Bb and Bc.

In step S340, the intrinsic mode function module 113 calculates multiple average envelope curves according to multiple upper envelope curves and multiple lower envelope curves respectively corresponding to the temporal intrinsic mode functions, and the intrinsic mode function module 113 averages the average envelope curves so as to generate a semi-intrinsic mode function (semi-IMF).

For example, in FIG. 4B, if the three temporal intrinsic mode functions are obtained by decomposing the first packets Ba, Bb and Bc in the above-mentioned step S330 and each temporal intrinsic mode function is provided with an upper envelope curve and a lower envelope curve, the intrinsic mode function module 113 calculates a first average envelop according to the upper envelope curve and the lower envelope curve of the first packet Ba, calculates a second average envelop according to the upper envelope curve and the lower envelope curve of the first packet Bb, and calculates a third average envelop according to the upper envelope curve and the lower envelope curve of the first packet Bc. Next, the intrinsic mode function module 113 further averages the first average envelop, the second average envelop and the third average envelop again, so as to generate a semi-intrinsic mode function.

More concretely, in one embodiment, a method of calculating the average envelop of each packet is as follows: the maxima and the minima of a sub-signal of each packet (such as the first packets Ba, Bb and Bc) can be searched by utilizing the intrinsic mode function module 113. According to the maxima, the minima and the signal length of the user physiological signal, an upper envelope curve and an lower envelope curve respectively corresponding to a sub-signal of each packet (such as the first packets Ba, Bb and Bc) are calculated by means of an interpolation method (for example, the upper envelope curve and the lower envelope curve of the sub-signal of the first packet Ba are calculated by utilizing the interpolation method). The average of the upper envelope curve and the lower envelope curve corresponding to the sub-signal of each packet (such as the first packets Ba, Bb and Bc) is calculated, so as to obtain the average envelope curves respectively corresponding to respective sub-signals of the first packets.

For example, the first average envelop curve is calculated according to the upper envelop curve and the lower envelop curve of the first packet Ba; the second average envelop curve is calculated according to the upper envelop curve and the lower envelop curve of the first packet Bb; and the third average envelop curve is calculated according to the upper envelop curve and the lower envelop curve of the first packet Bc. Therefore, the first average envelop curve, the second average envelop curve and the third average envelop curve can further be averaged again, so as to generate a semi-intrinsic mode function.

In one embodiment, the step S340 further includes the steps that after generating semi-intrinsic mode function, the intrinsic mode function module 113 adds a first constant (such as 1) to the first box number (such as 3) so as to generate a second box number (such as 4), and the step S310 is executed again, so that the packeting module 111 separates the user physiological signal into multiple second packets according to the second box number. For example, as shown in FIG. 4B, the user physiological signal is separated into four packets, and in this embodiment, the four packets can be referred to as second packets since the step S310 is executed for the second time. Next, the empirical mode decomposition module 112 respectively performs the sifting process on the second packets by utilizing the empirical mode decomposition so as to obtain the temporal intrinsic mode functions respectively corresponding to the second packets, continues to execute steps S350-S360 according to the temporal intrinsic mode functions, so as to determine whether another main component section can be extracted on the conditions that the user physiological signal is separated into multiple second packets according to the second box number.

In another embodiment, the packet numbers can be sequentially regulated. For example, each time the step S340 is executed, the packet number is larger than when the step S340 is executed last time by 1. Therefore, different packet numbers can be substituted into the step S340 in sequence according to their values, and then steps S350-S360 are executed respectively, so as to respectively determine whether other main component sections can be extracted corresponding to each packet number on the conditions of different packet numbers.

In step S350, the main component module 114 calculates at least one correlation coefficient according to the semi-intrinsic mode function and at least another semi-intrinsic mode function.

For example, the main component module 114 calculates at least one correlation coefficient by utilizing at least another semi-intrinsic mode function (for example, the another semi-intrinsic mode function is generated on the conditions that the second box number is 4 or 2) that has the adjacent packet number together with the semi-intrinsic mode function (for example, the semi-intrinsic mode function is generated on the conditions that the first box number is 3).

On the other hand, in one embodiment, another semi-intrinsic mode function is correlated with the second box number, and the difference between the second box number (such as 4) and the first box number (such as 3) is smaller than a second constant (such as 1). Herein, the first box number and the second box number are not smaller than zero.

In step S360, when the at least one correlation coefficient is larger than a correlation coefficient threshold, the main component module 114 determines at least one signal section corresponding to the at least one correlation coefficient as at least one main component section of the user physiological signal.

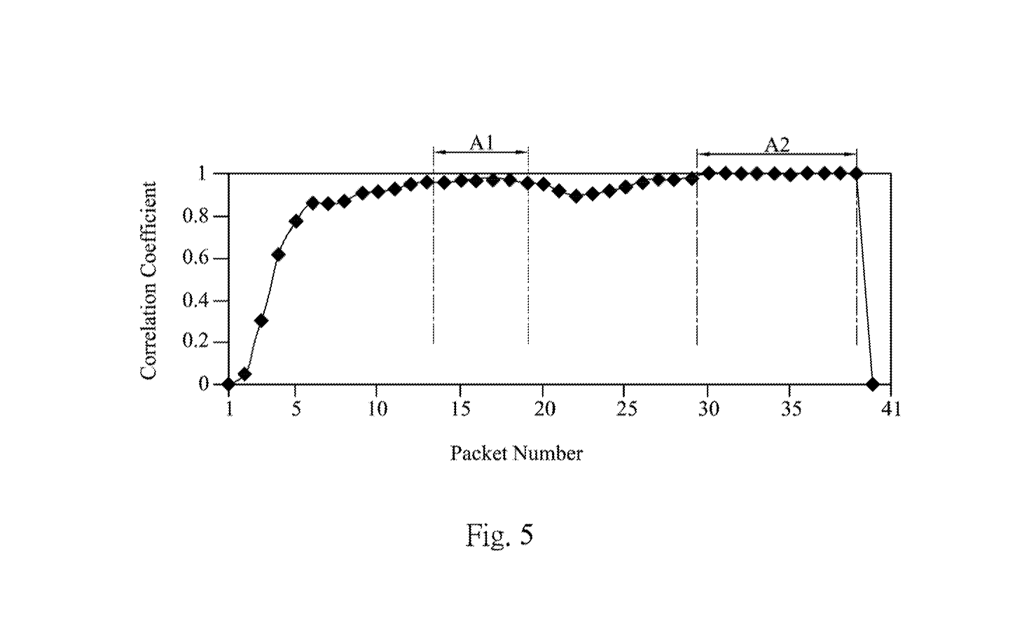

For example, as shown in FIG. 5, FIG. 5 illustrates a schematic view of a correlation coefficient according to an embodiment of the present invention. When the at least one correlation coefficient (for example, in FIG. 5, when the packet number is 18, the correlation coefficient is 0.99) is larger than a correlation coefficient threshold (such as 0.98), the main component module 114 determines at least one signal section A1 corresponding to the at least one correlation coefficient (0.99) as at least one main component section of the user physiological signal. In one embodiment, as can be seen from FIG. 5, the signal sections A1 and A2 have many correlation coefficients larger than the correlation coefficient threshold (such as 0.98), and thus the signal sections A1 and A2 can be regarded as main component sections.

In another embodiment, the main component module 114 determines one of at least one correlation coefficient with the maximum in the main component section A2 as a main component signal, and records a main component packet number corresponding to the main component signal. Herein, the main component signal is correlated with a reflection waveform, an incident waveform, a chest exercise and an abdominal exercise.

For example, in FIG. 5, when the packet number of the main component section A2 is 40, the main component section A2 has the maximal correlation coefficient (0.995), and thus the packeting module 111 can set the main component packet number as 40, determine a main component signal according to the main component packet number and record the packet number.

Therefore, the physiological signal measuring system 100 can be used to extract the main component signal, so as to judge users' physiological conditions. Besides, the physiological signal measuring system 100 can be applied to hardware facilities in the aspect of home care, so that the physical facilities have a function of timely diagnosing patients' conditions and give health indicators about users' current physical conditions after performing real-time treatment and analysis on users' breathing and blood pressure signals, or a medical robot is directly arranged at the rear end to directly make a diagnosis to achieve a function of far-end nursing.

On the other hand, the above-mentioned method can also be implemented in an application program of an intelligent product, so as to let users learn about their health conditions anytime anywhere and provide users with instant health information and appropriate health policies. In addition, the above-mentioned method can also be applied to an intelligent product, so as to let users record their physical conditions and exercise progress during the exercise.

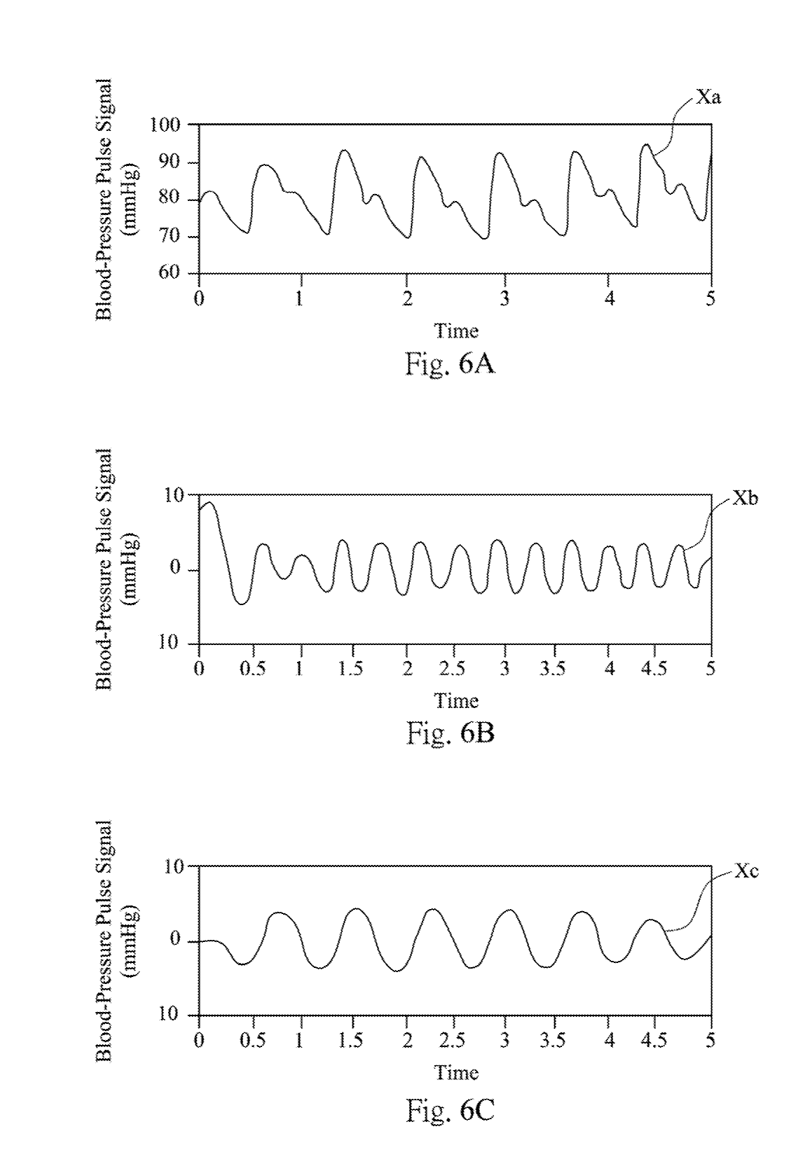

As shown in FIGS. 6A-6C, FIGS. 6A-6C illustrate schematic views of a mode of application of the physiological signal measuring system 100 according to an embodiment of the present invention. In this embodiment, the user physiological signal is a blood-pressure pulse signal Xa. After the blood-pressure pulse signal Xa is processed through the above-mentioned steps of FIG. 3, the main component sections A1 and A2 similarly as shown in FIG. 5 can be obtained, and the main component module 114 respectively determines the maximal correlation coefficients in the component sections A1 and A2 as main component signals. For example, the main component signal of the main component section A1 is a reflection waveform Xb, and the main component signal of the main component section A2 is an incident waveform Xc. In one embodiment, the physiological signal measuring system 100 can store the main component signal (such as the reflection waveform Xb and/or the incident waveform Xc), so as to record main component distribution conditions corresponding to an individual user.

In one embodiment, by means of the method for extracting the main component signal and the main component packet number, each time users need to perform measurement or real-time monitoring, they only need to input the main component signal and the main component packet number to the physiological signal measuring system 100, and the physiological signal measuring system 100 can perform real-time decomposition according to the distribution locations of normal main components measured by the users last time.

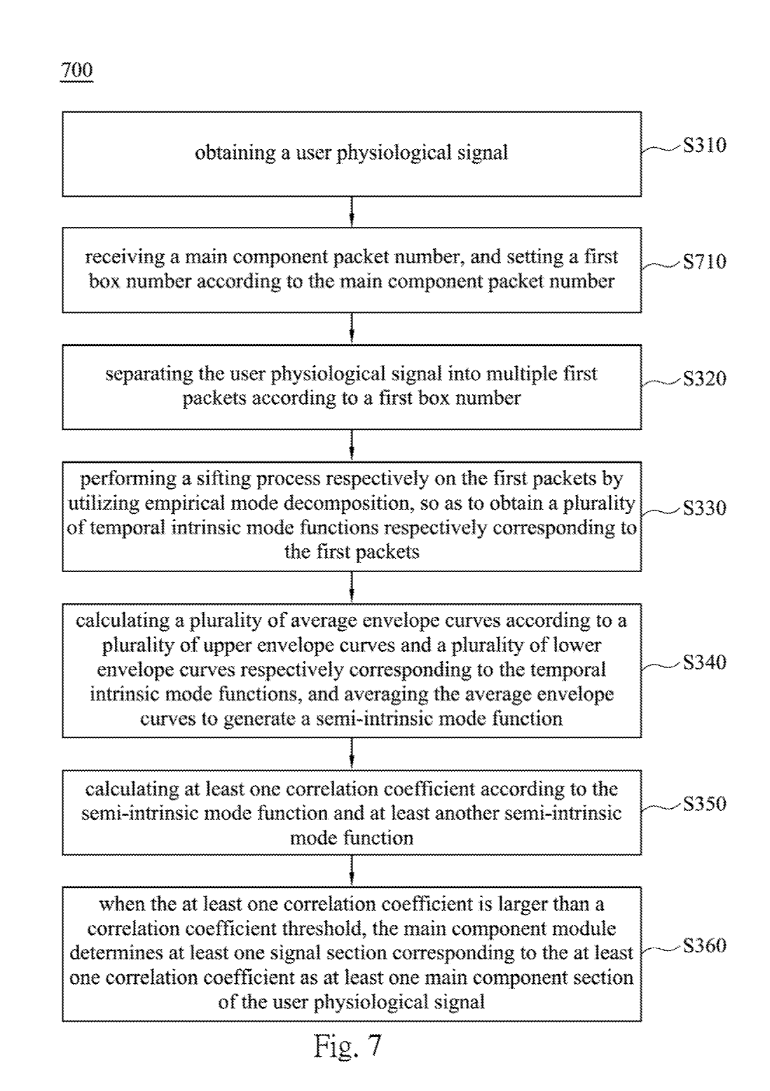

In one embodiment, referring to FIG. 7, FIG. 7 illustrates a flow diagram of a physiological signal measuring method 700 according to an embodiment of the present invention. FIG. 7 is different from FIG. 3 in that FIG. 7 further includes step S710. The remaining steps are the same as FIG. 3, and thus it is not repeated herein.

In step S710, the packeting module 111 is further used for receiving a main component packet number, and setting a first box number according to the main component packet number. For example, in FIG. 5, on the conditions that the reflection waveform Xb of the main component signal can be obtained when it is learnt that the packet number is 18 by means of the aforesaid physiological signal measuring method 300, in step S710, the packeting module 111 can set the main component packet number as 18, also set the first box number as 18 according to the main component packet number, and execute the follow-up Step S320-S360 according to the first box number.

Therefore, after the positions of main components of the user physiological signal of some user are found, the user physiological signal can be decomposed directly according to the main component packet number (for example, the main component packet number is 18) without needing to be decomposed one by one in sequence according to different packet numbers; that is, by means of the aforesaid method, the user physiological signal does not need to be decomposed respectively when the packet number is 1, 2, 3 . . . . Therefore, the user physiological signal can be directly decomposed by utilizing the obtained main component packet number, so as to greatly reduce the calculated amount in the decomposition process through a conventional method.

The present invention provides the method for physiological signal measurement by means of parallelization and the system thereof. The sifting process is carried out after the packeting process is performed on the user physiological signal; the aforesaid correlated method for calculating the average envelops is applied; the processed packets are averaged so as to serve as a temporal intrinsic mode function; the positions of main components are determined by means of the correlation coefficient so as to extract the main components; and all these processes can be calculated through parallelization. For example, the multi-core processor is adopted to calculate the aforesaid correlated steps of processing each first packet in parallel, so as to decompose continuous blood pressure pulse and respiratory movement signals, extract main components more quickly, be applied more conveniently to related hardware for future real-time processing and improve further integration of medicine and healthy life.

Although the present invention has been disclosed with reference to the embodiments, these embodiments are not intended to limit the present invention. Various modifications and variations can be made by those of skills in the art without departing from the spirit and scope of the present invention, and thus the protection scope of the present invention shall be defined by the appended claims.

What is claimed is:

1. A physiological signal measuring system, comprising:
a processor, comprising:
a packeting module for obtaining a user physiological signal and separating the user physiological signal into a plurality of first packets according to a first box number;
an empirical mode decomposition module for performing a sifting process respectively on the first packets by utilizing empirical mode decomposition (EMD), so as to obtain a plurality of temporal intrinsic mode functions (temporal IMFs) respectively corresponding to the first packets;
an intrinsic mode function module for calculating a plurality of average envelope curves according to a plurality of upper envelope curves and a plurality of lower envelope curves respectively corresponding to the temporal intrinsic mode functions, and averaging the average envelope curves to generate a semi-intrinsic mode function (semi-IMF); and
a main component module for calculating at least one correlation coefficient according to the semi-intrinsic mode function and at least another semi-intrinsic mode function, wherein when the at least one correlation coefficient is larger than a correlation coefficient threshold, the main component module determines at least one signal section corresponding to the at least one correlation coefficient as at least one main component section of the user physiological signal.

2. The physiological signal measuring system of claim 1, further comprising:
a sensor for measuring an initial physiological signal which is an analog signal; and
an analog digital converter for converting the initial physiological signal into a user physiological signal which is a digital signal.

3. The physiological signal measuring system of claim 1, further comprising:
a stop criteria setting module for judging whether a sifting result of the sifting process meets a stop criteria; wherein, the sifting result corresponds to one of the first packets;
if the stop criteria setting module judges that the sifting result of the sifting process meets the stop criteria, one of the temporal intrinsic mode functions is generated; and
if the stop criteria setting module judges that the sifting result of the sifting process does not meet the stop criteria, the sifting result is substituted into the empirical mode decomposition to perform the sifting process again.

4. The physiological signal measuring system of claim 1, wherein the intrinsic mode function module searches for a maxima and a minima of a sub-signal of each one of the first packets, calculates the upper envelope curve and the lower envelope curve respectively corresponding to a sub-signal of each one of the first packets by means of an interpolation method according to the maxima, the minima and the signal length of the user physiological signal, and calculates the average of the upper envelope curve and the lower envelope curve corresponding to the sub-signal of each one of the first packets, so as to obtain the average envelope curves respectively corresponding to respective sub-signals of the first packets.

5. The physiological signal measuring system of claim 1, wherein:
after generating the semi-intrinsic mode function, the intrinsic mode function module adds a first constant to the first box number, so as to generate a second box number; and
the packeting module separates the user physiological signal into a plurality of second packets according to the second box number; and the empirical mode decomposition module performs the sifting process respectively on the second packets by utilizing the empirical mode decomposition, so as to obtain the temporal intrinsic mode functions respectively corresponding to the second packets.

6. The physiological signal measuring system of claim 1, wherein the another semi-intrinsic mode function is correlated with a second box number, and the difference between the second box number and the first box number is smaller than a second constant.

7. The physiological signal measuring system of claim 1, wherein after the packeting module divides the user physiological signal into a plurality of section signals sequentially, the first packets are formed by performing sequential decimation according to the section signals.

8. The physiological signal measuring system of claim 1, wherein the packeting module is further used for receiving a main component packet number and setting the first box number according to the main component packet number.

9. The physiological signal measuring system of claim 1, wherein the main component module determines a maximum one of the at least one correlation coefficient of the at least one main component section as a main component signal, and records a main component packet number corresponding to the main component signal.

10. The physiological signal measuring system of claim 9, wherein the main component signal is correlated with a reflection waveform, an incident waveform, a chest exercise and an abdominal exercise.

11. A physiological signal measuring method, comprising:
obtaining a user physiological signal and separating the user physiological signal into a plurality of first packets according to a first box number;
performing a sifting process respectively on the first packets by utilizing empirical mode decomposition (EMD), so as to obtain a plurality of temporal intrinsic mode functions (temporal IMFs) respectively corresponding to the first packets;
calculating a plurality of average envelope curves according to a plurality of upper envelope curves and a plurality of lower envelope curves respectively corresponding to the temporal intrinsic mode functions, and averaging the average envelope curves to generate a semi-intrinsic mode function (semi-IMF);
calculating at least one correlation coefficient according to the semi-intrinsic mode function and at least another semi-intrinsic mode function; and
when the at least one correlation coefficient is larger than a correlation coefficient threshold, determining at least one signal section corresponding to the at least one correlation coefficient as at least one main component section of the user physiological signal.

12. The physiological signal measuring method of claim 11, further comprising:
measuring an initial physiological signal which is an analog signal; and
converting the initial physiological signal into the user physiological signal which is a digital signal.

13. The physiological signal measuring method of claim 11, further comprising:
judging whether a sifting result of the sifting process meets one stop criteria, wherein the sifting result corresponds to one of the first packets;
if it is judged that the sifting result of the sifting process meets the stop criteria, generating one of the temporal intrinsic mode functions; and
if it is judged by the stop criteria setting module that the sifting result of the sifting process does not meet the stop criteria, substituting the sifting result into the empirical mode decomposition to perform the sifting process again.

14. The physiological signal measuring method of claim 13, further comprising:
searching for a maxima and a minima of a sub-signal of each one of the first packets; calculating the upper envelope curve and the lower envelope curve respectively corresponding to a sub-signal of each one of the first packets by means of an interpolation method according to the maxima, the minima and the signal length of the user physiological signal; and
calculating the average of the upper envelope curve and the lower envelope curve corresponding to the sub-signal of each one of the first packets, so as to obtain the average envelope curves respectively corresponding to respective sub-signals of the first packets.

15. The physiological signal measuring method of claim 14, further comprising:
after the semi-intrinsic mode function is generated, adding a first constant to the first box number, so as to generate a second box number,
separating the user physiological signal into a plurality of second packets according to the second box number; and
respectively performing the sifting process on the second packets by utilizing the empirical mode decomposition, so as to obtain the temporal intrinsic mode functions respectively corresponding to the second packets.

16. The physiological signal measuring method of claim 11, wherein the another semi-intrinsic mode function is correlated with a second box number, and the difference between the second box number and the first box number is smaller than a second constant.

17. The physiological signal measuring method of claim 11, further comprising:
after the user physiological signal is sequentially divided into a plurality of section signals, forming the first packets by performing sequential decimation according to the section signals.

18. The physiological signal measuring method of claim 14, further comprising:
receiving a main component packet number and setting the first box number according to the main component packet number.

19. The physiological signal measuring method of claim 11, further comprising:
determining a maximum one of the at least one correlation coefficient of the at least one main component section as a main component signal, and recording a main component packet number corresponding to the main component signal.

20. The physiological signal measuring method of claim 19, wherein the main component signal is correlated with a reflection waveform, an incident waveform, a chest exercise and an abdominal exercise.
